(19) United States
(12) Patent Application Publication
(10) Pub. No.: US 2020/0010481 A1
(43) Pub. Date: Jan. 9, 2020

(54) SOLID STATE FORMS OF MIDOSTAURIN

(71) Applicant: TEVA PHARMACEUTICAL WORKS LTD., Debrecen (HU)

(72) Inventors: Zsuzsa Potarine Juhasz, Debrecen (HU); Csilla Nemethe Racz, Debrecen (HU); Szabolcs Struba, Balmazujvaros (HU); Renata Kertine Ferenczi, Hajdusamson-Samsonkert (HU); Tivadar Tamas, Debrecen (HU)

(21) Appl. No.: 16/491,225

(22) PCT Filed: Mar. 6, 2018
(86) PCT No.: PCT/US18/21015

§ 371 Date: Sep. 5, 2019

Related U.S. Application Data
(60) Provisional application No. 62/467,643, filed on Mar. 6, 2017.
(60) Provisional application No. 62/484,426, filed on Apr. 12, 2017.
(60) Provisional application No. 62/489,679, filed on Apr. 25, 2017.
(60) Provisional application No. 62/512,912, filed on May 31, 2017.
(60) Provisional application No. 62/618,909, filed on Jan. 18, 2018.

(51) Int. Cl. C07D 498/22

(52) U.S. Cl. CPC C07D 498/22; C07B 2200/13

(57) ABSTRACT

The present disclosure relates to solid state forms of Midostaurin, processes for preparation thereof, pharmaceutical compositions thereof, and use thereof in the treatment of Acute Myelogenous Leukemia (AML) and Aggressive Systemic Mastocytosis. Chemical SHNCH formula: C₃₅H₃₀N₄O₄ Molecular mass: 570.64 g/mol

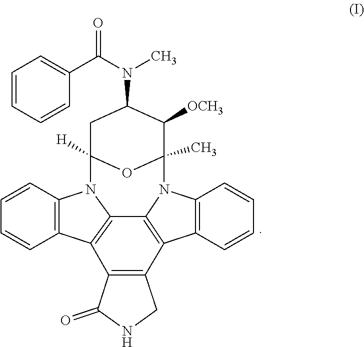 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4]

### FIELD OF THE INVENTION

The present disclosure relates to solid state forms of Midostaurin, processes for preparation thereof and pharmaceutical compositions thereof.

### BACKGROUND OF THE INVENTION

Midostaurin has the chemical name N-[(9S,10R, 11R,13R)-2,3,10,11,12,13-Hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide and has the following chemical structure:

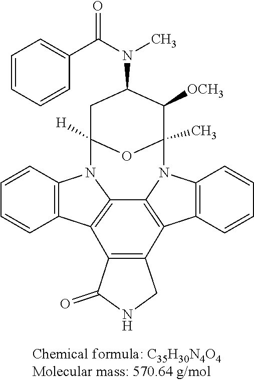 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4]

Midostaurin is being developed for the treatment of Acute Myelogenous Leukemia (AML) and Aggressive Systemic Mastocytosis.

Midostaurin is disclosed in U.S. Pat. No. 5,093,330.

U.S. Pat. No. 8,198,435 discloses crystalline Midostaurin Form II, U.S. Pat. No. 9,150,589 discloses crystalline Midostaurin Form III and US2015/0368268 discloses crystalline Midostaurin Form IV.

Polymorphism, the occurrence of different crystal forms, is a property of some molecules and molecular complexes. A single compound, like Midostaurin, may give rise to a variety of polymorphs having distinct crystal structures and physical properties like melting point, thermal behaviors (e.g. measured by thermogravimetric analysis—“TGA”, or differential scanning calorimetry—“DSC”), X-ray powder diffraction (XRPD) pattern, infrared absorption fingerprint, Raman absorption fingerprint, and solid state (¹³C-) NMR spectrum. One or more of these techniques may be used to distinguish different polymorphic forms of a compound.

Different solid state forms (including solvated forms) of an active pharmaceutical ingredient may possess different properties. Such variations in the properties of different solid state forms and solvates may provide a basis for improving formulation, for example, by facilitating better processing or handling characteristics, improving the dissolution profile, or improving stability (polymorph as well as chemical stability) and shelf-life. These variations in the properties of different solid state forms may also provide improvements to the final dosage form, for instance, if they serve to improve bioavailability. Different solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new solid state forms and solvates of a pharmaceutical product can provide materials having desirable processing properties, such as ease of handling, ease of processing, storage stability, and ease of purification or as desirable intermediate crystal forms that facilitate conversion to other polymorphic forms. New polymorphic forms and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life.

For at least these reasons, there is a need for additional solid state forms (including solvated forms) of Midostaurin.

### SUMMARY OF THE INVENTION

The present disclosure relates to solid state forms of Midostaurin, processes for preparation thereof, and pharmaceutical compositions comprising these solid state forms.

The present disclosure also provides uses of the solid state forms of Midostaurin for preparing other solid state forms of Midostaurin.

The present disclosure further provides processes for preparing other solid state forms of Midostaurin.

In another embodiment, the present disclosure encompasses the described solid state forms of Midostaurin for uses in the preparation of pharmaceutical compositions and/or formulations, optionally for the treatment of carcinoid syndrome or for the treatment of Acute Myelogenous Leukemia (AML) and Aggressive Systemic Mastocytosis.

In another embodiment, the present disclosure encompasses uses of the described solid state form of Midostaurin for the preparation of pharmaceutical compositions and/or formulations.

The present disclosure further provides pharmaceutical compositions comprising any one or a combination of the solid state form of Midostaurin according to the present disclosure.

In yet another embodiment, the present disclosure encompasses pharmaceutical formulations comprising any one or a combination of the described solid state forms of Midostaurin and at least one pharmaceutically acceptable excipient.

The present disclosure encompasses processes to prepare said pharmaceutical formulations of Midostaurin comprising combining any one or a combination of the described solid state forms and at least one pharmaceutically acceptable excipient.

The solid state forms defined herein as well as the pharmaceutical compositions or formulations of the solid state form of Midostaurin can be used as medicaments, particularly for the treatment of Acute Myelogenous Leukemia (AML) and Aggressive Systemic Mastocytosis.

The present disclosure also provides methods of treating Acute Myelogenous Leukemia (AML) and Aggressive Systemic Mastocytosis comprising administering a therapeutically effective amount of any one or a combination of the described solid state forms of the present disclosure, or at least one of the herein described pharmaceutical compositions or formulations, to a subject suffering from Acute Myelogenous Leukemia (AML) and Aggressive Systemic Mastocytosis, or otherwise in need of the treatment.

The present disclosure also provides uses of the solid state forms of Midostaurin of the present disclosure, or at least one of the above pharmaceutical compositions or formulations for the manufacture of medicaments Acute Myelogenous Leukemia (AML) and Aggressive Systemic Mastocytosis.

### BRIEF DESCRIPTION OF THE FIGURES

FIG. 1 shows an X-ray powder diffractogram (XRPD) of amorphous Midostaurin.

FIG. 2 shows an X-ray powder diffractogram (XRPD) of crystalline form V of Midostaurin.

FIG. 3 shows an X-ray powder diffractogram (XRPD) of crystalline form VI of Midostaurin.

FIG. 4 shows an X-ray powder diffractogram (XRPD) of crystalline form VII of Midostaurin.

FIG. 5 shows an X-ray powder diffractogram (XRPD) of crystalline form VIII of Midostaurin.

FIG. 6 shows an X-ray powder diffractogram (XRPD) of crystalline form IX of Midostaurin.

FIG. 7 shows an X-ray powder diffractogram (XRPD) of crystalline form X of Midostaurin.

FIG. 8 shows an X-ray powder diffractogram (XRPD) of crystalline form XI of Midostaurin.

FIG. 9 shows an X-ray powder diffractogram (XRPD) of crystalline form XII of Midostaurin.

FIG. 10 shows an X-ray powder diffractogram (XRPD) of crystalline form XIII of Midostaurin.

FIG. 11 shows an X-ray powder diffractogram (XRPD) of purified Midostaurin obtained in Example 2a.

FIG. 12 shows an X-ray powder diffractogram (XRPD) of purified Midostaurin obtained in Example 2b.

FIG. 13 shows an X-ray powder diffractogram (XRPD) of crystalline form XIV of Midostaurin.

FIG. 14 shows an X-ray powder diffractogram (XRPD) of crystalline form XV of Midostaurin.

FIG. 15 shows an X-ray powder diffractogram (XRPD) of crystalline form VI of Midostaurin obtained in example 5a.

FIG. 16 shows an X-ray powder diffractogram (XRPD) of crystalline form XVI of Midostaurin.

FIG. 17 shows an FT-IR spectra of crystalline form VI of Midostaurin

### DETAILED DESCRIPTION OF THE INVENTION

The present disclosure relates to solid state forms of Midostaurin, processes for preparation thereof and pharmaceutical compositions thereof.

The disclosure also relates to the conversion of the described solid state form of Midostaurin to other solid state forms of Midostaurin.

The solid state forms of Midostaurin and according to the present disclosure may have advantageous properties selected from at least one of: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Midostaurin or referred to herein as being characterized by graphical data “as depicted in” a Figure will thus be understood to include any crystal forms of the Midostaurin or, characterized with the graphical data having such small variations, as are well known to the skilled person, in comparison with the Figure.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or 0% of any other forms of the subject compound as measured, for example, by XRPD. Thus, the solid state form of Midostaurin described herein as is substantially free of any other solid state forms would be understood to contain greater than about 80% (w/w), greater than about 90% (w/w), greater than about 95% (w/w), greater than about 98% (w/w), greater than about 99% (w/w), or 100% of the subject solid state form of Midostaurin. Accordingly, in some embodiments of the disclosure, the described solid state forms of Midostaurin and may contain from about 1% to about 20% (w/w), from about 5% to about 20% (w/w), or from about 5% to about 10% (w/w) of one or more other solid state forms of Midostaurin.

As used herein, unless stated otherwise, XRPD peaks reported herein are optionally measured using CuK_{α} radiation, λ=1.54 Å.

As used herein, the term “isolated” in reference to solid state forms of Midostaurin of the present disclosure corresponds to solid state form of Midostaurin that is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, about 10 to about 18 hours, or about 16 hours.

As used herein, the expression “wet crystalline form” refers to a polymorph that was not dried using any conventional techniques to remove residual solvent. Examples for such conventional techniques can be, but not limited to, evaporation, vacuum drying, oven drying, drying under nitrogen flow, etc.

As used herein, the expression “dry crystalline form” refers to a polymorph that was dried using any conventional techniques to remove residual solvent. Examples of such conventional techniques can be, but are not limited to, evaporation, vacuum drying, oven drying, drying under nitrogen flow, etc.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of from about 10 mbar to 50 mbar.

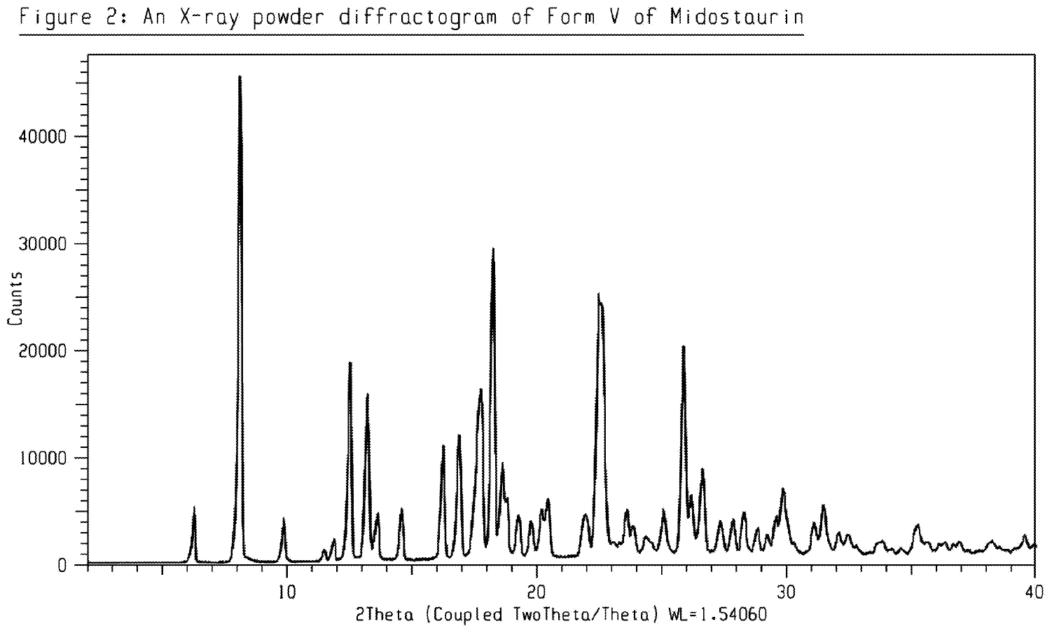

The present disclosure comprises a crystalline form of Midostaurin designated as Form V. The crystalline Form V of Midostaurin can be characterized by data selected from one or more of the following: an XRPD pattern having peaks at 8.1, 12.5, 13.2, 16.2 and 18.2 degrees 2-theta±0.2 degrees 2-theta; an XRPD pattern as depicted in FIG. 2; and combinations of these data.

Crystalline Form V of Midostaurin may be further characterized by the XRPD pattern having peaks at 8.1, 12.5, 13.2, 16.2 and 18.2 degrees 2-theta±0.2 degrees 2-theta, and also having one, two, three, four, five or six additional peaks selected from 6.2, 9.8, 16.8, 17.7, 22.5 and 25.8 degrees 2-theta±0.2 degrees 2-theta.

Crystalline Form V of Midostaurin may be characterized by each of the above characteristics alone/or by all possible combinations, e.g. by XRPD pattern having peaks at 8.1, 12.5, 13.2, 16.2 and 18.2 degrees 2-theta±0.2 degrees 2-theta and an XRPD pattern as depicted in FIG. 2. The present disclosure also provides the use of the solid state form of Midostaurin for preparing other solid state forms of Midostaurin and solid state forms thereof.

Crystalline Form V of Midostaurin may be a solvate. Crystalline Form V may be an Ethyl-acetate-solvate.

PXRD peak list for crystalline Form V of Midostaurin is shown in table 1.

TABLE 1
peak position (degrees two theta ±
0.2 degrees two-theta)

6.2
8.1
9.8
12.5
13.2
13.6
14.5
16.2
16.8
17.7
18.2
18.6
19.2
19.7
22.5
25.0
25.8
26.1

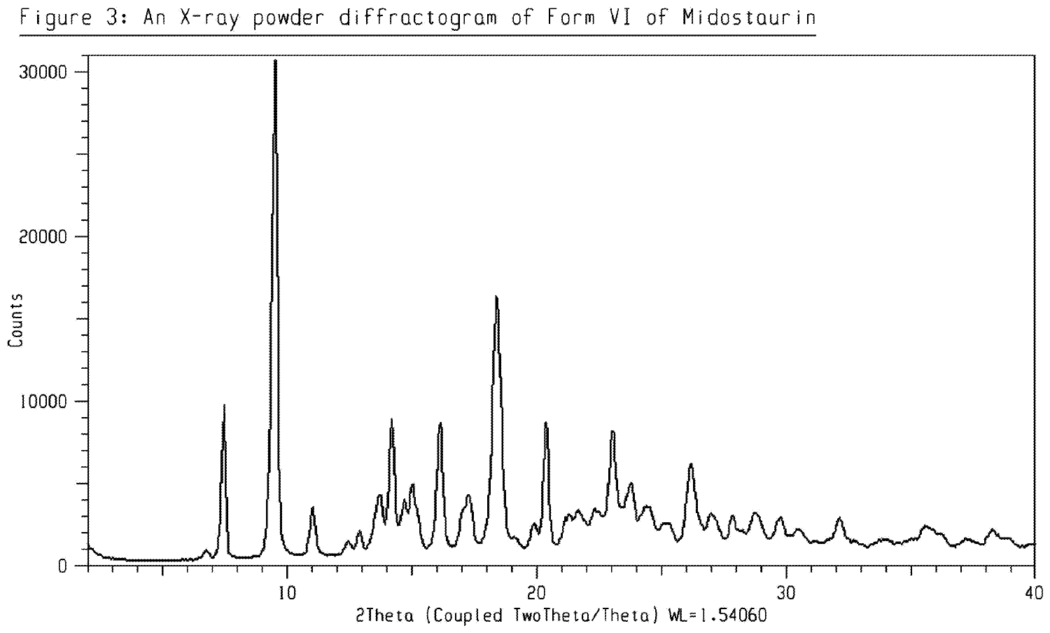

The present disclosure also comprises a crystalline form of Midostaurin designated as Form VI. The crystalline Form VI of Midostaurin can be characterized by data selected from one or more of the following: an XRPD pattern having peaks at 7.5, 9.5, 11.0, 14.2 and 16.1 degrees 2-theta±0.2 degrees 2-theta; an XRPD pattern as depicted in FIG. 3; and combinations of these data.

Crystalline Form VI of Midostaurin may be further characterized by the XRPD pattern having peaks at 7.5, 9.5, 11.0, 14.2 and 16.1 degrees 2-theta±0.2 degrees 2-theta, and also having one, two, three, four or five additional peaks selected from 17.3, 18.4, 20.4, 23.0, and 26.1 degrees 2-theta±0.2 degrees 2-theta.

Crystalline Form VI of Midostaurin may be characterized by each of the above characteristics alone/or by all possible combinations, e.g. by XRPD pattern having peaks at 7.5, 9.5, 11.0, 14.2 and 16.1 degrees 2-theta±0.2 degrees 2-theta; and an XRPD pattern as depicted in FIG. 3. The present disclosure also provides the use of the solid state form of Midostaurin for preparing other solid state forms of Midostaurin and solid state forms thereof.

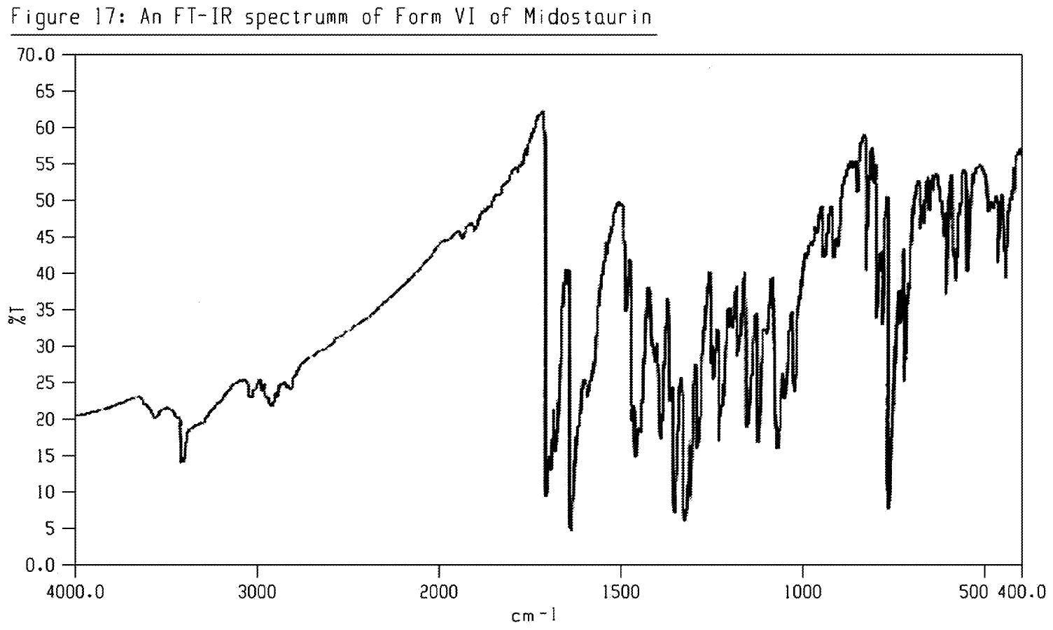

Crystalline form VI of Midostaurin may be further characterized by data selected from one of the following: an FT-IR spectrum having one, two, three, four or more peaks selected from 3428, 1701, 1635, 1454, 1345, 1223, 1115, 1064, 823, 750 and 713 cm⁻¹±1 cm⁻¹; an FT-IR spectrum as depicted in FIG. 17, and combinations of these data.

Crystalline Form VI of Midostaurin can be further characterized by TGA weight loss between 1.0-5.0% up to about 130° C.

Crystalline Form VI of Midostaurin is a hydrate. The water content in crystalline Form VI may be determined by TGA and/or Karl Fischer. The water content in crystalline Form VI may be between 1.0% to 5.0%, between 1.2% to 4.5%, between 1.3% to 4.0%, between 1.6% to 3.3%, between 1.6% to 2.5%. Preferably, the water content in crystalline Form VI is about 2%.

PXRD peak list for crystalline Form VI of Midostaurin is shown in table 2.

TABLE 2
peak position (degrees two theta ±
0.2 degrees two-theta)

6.8
7.5
9.5
11.0
12.5
12.9
13.7
14.2
14.7
15.0
16.1
17.3
18.4
19.9
20.4
21.2
22.3
23.0
23.7
24.5
25.0
26.1
26.9
27.8
28.7
29.7
30.4
32.1

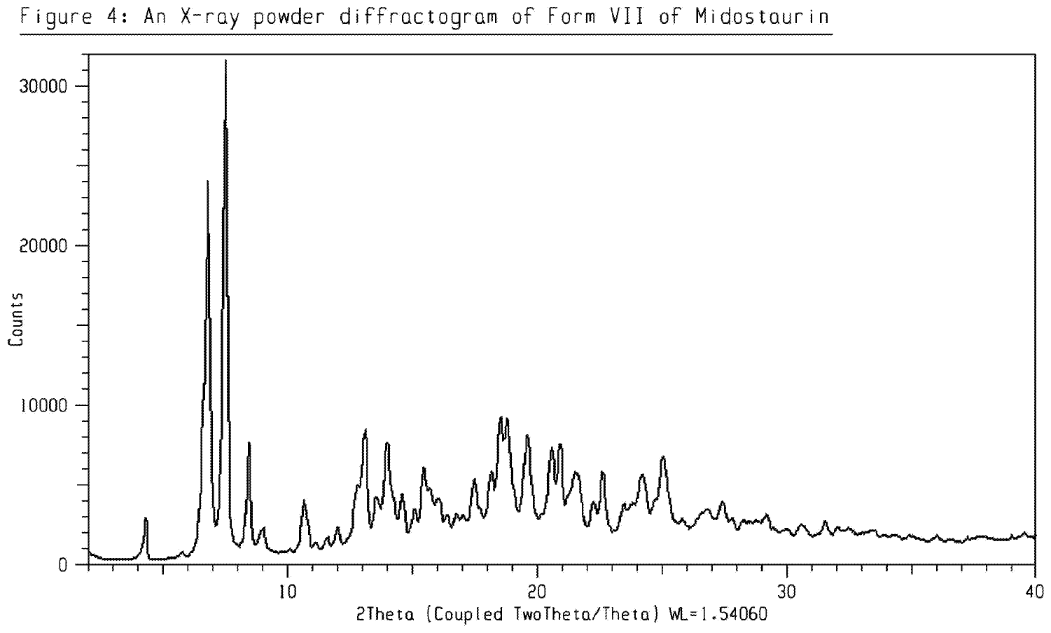

The present disclosure comprises a crystalline form of Midostaurin designated as Form VII. The crystalline Form VII of Midostaurin can be characterized by data selected from one or more of the following: an XRPD pattern having peaks at 4.3, 6.8, 7.5, 8.5 and 10.7 degrees 2-theta±0.2 degrees 2-theta; an XRPD pattern as depicted in FIG. 4; and combinations of these data.

Crystalline Form VII of Midostaurin may be further characterized by the XRPD pattern having peaks at 4.3, 6.8, 7.5, 8.5 and 10.7 degrees 2-theta±0.2 degrees 2-theta, and also having one, two, three, four or five additional peaks selected from 13.1, 14.0, 19.6, 22.7, and 25.1 degrees 2-theta±0.2 degrees 2-theta.

Crystalline Form VII of Midostaurin may be characterized by each of the above characteristics alone/or by all possible combinations, e.g. by XRPD pattern having peaks at 4.3, 6.8, 7.5, 8.5 and 10.7 degrees 2-theta±0.2 degrees 2-theta and an XRPD pattern as depicted in FIG. 4. The present disclosure also provides the use of the solid state form of Midostaurin for preparing other solid state forms of Midostaurin and solid state forms thereof.

Crystalline Form VII of Midostaurin may be a solvate. Crystalline Form VII may be a Methyl-isobutyl-ketone solvate.

PXRD peak list for crystalline Form VII of Midostaurin is shown in table 3.

TABLE 3
peak position (degrees two theta ±
0.2 degrees two-theta)

4.3
6.8
7.5
8.5
9.1
10.7
13.1
14.0
14.6
15.1
15.5
16.1
17.5
18.2
18.6
18.8
19.6
20.6
21.0
21.6
22.3
22.7
23.5
24.3
25.1
26.7
27.5

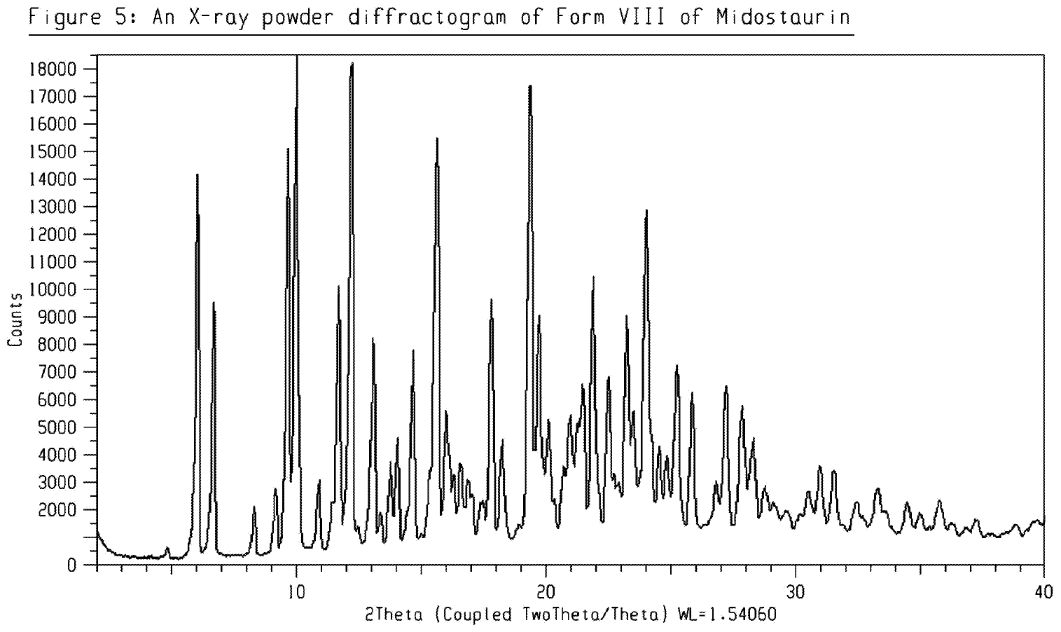

The present disclosure comprises a crystalline form of Midostaurin designated as Form VIII. The crystalline Form VIII of Midostaurin can be characterized by data selected from one or more of the following: an XRPD pattern having peaks at 6.0, 6.6, 9.6, 10.0 and 12.1 degrees 2-theta±0.2 degrees 2-theta; an XRPD pattern as depicted in FIG. 5; and combinations of these data.

Crystalline Form VIII of Midostaurin may be further characterized by the XRPD pattern having peaks at 6.0, 6.6, 9.6, 10.0 and 12.1 degrees 2-theta±0.2 degrees 2-theta, and also having one, two, three, four or five additional peaks selected from 11.6, 13.0, 14.6, 15.6, and 19.3 degrees 2-theta±0.2 degrees 2-theta.

Crystalline Form VIII of Midostaurin may be characterized by each of the above characteristics alone/or by all possible combinations, e.g. by XRPD pattern having peaks at 6.0, 6.6, 9.6, 10.0 and 12.1 degrees 2-theta±0.2 degrees 2-theta and an XRPD pattern as depicted in FIG. 5. The present disclosure also provides the use of the solid state form of Midostaurin for preparing other solid state forms of Midostaurin and solid state forms thereof.

Crystalline Form VIII of Midostaurin may be a solvate. Crystalline Form VIII may be a 4-heptanone solvate.

PXRD peak list for crystalline Form VIII of Midostaurin is shown in table 4.

TABLE 4
peak position (degrees two theta ±
0.2 degrees two-theta)

6.0
6.6
8.3
9.1
9.6
10.0
10.8
11.6
12.1
13.0
13.7
14.0
14.6
15.6
16.0
16.5
17.8
18.2
19.3
19.7
20.1
20.9
21.4
21.8
22.5
23.2
23.5
24.0
24.5
24.8
25.2
25.8
26.8
27.1
27.8
28.2
28.7
30.4
30.9
31.5
32.4
33.2

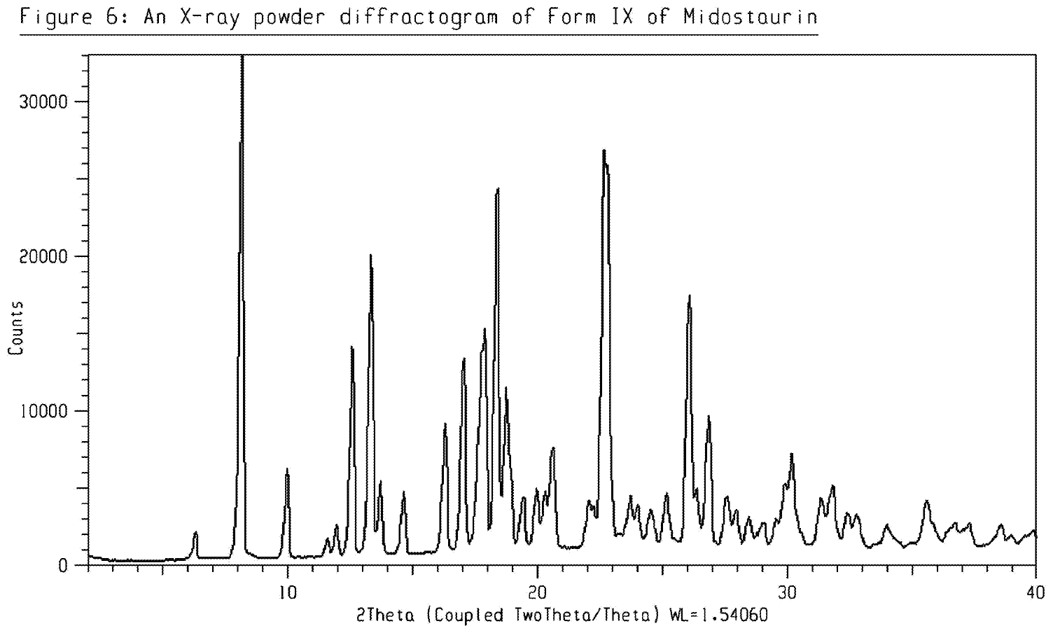

The present disclosure comprises a crystalline form of Midostaurin designated as Form IX. The crystalline Form IX of Midostaurin can be characterized by data selected from one or more of the following: an XRPD pattern having peaks at 8.1, 9.9, 12.6, 13.3 and 14.6 degrees 2-theta±0.2 degrees 2-theta; an XRPD pattern as depicted in FIG. 6; and combinations of these data.

Crystalline Form IX of Midostaurin may be further characterized by the XRPD pattern having peaks at 8.1, 9.9, 12.6, 13.3 and 14.6 degrees 2-theta±0.2 degrees 2-theta, and also having one, two, three, four or five additional peaks selected from 16.3, 17.0, 17.8, 18.4 and 26.1 degrees 2-theta±0.2 degrees 2-theta.

Crystalline Form IX of Midostaurin may be characterized by each of the above characteristics alone/or by all possible combinations, e.g. by XRPD pattern having peaks at 8.1, 9.9, 12.6, 13.3 and 14.6 degrees 2-theta±0.2 degrees 2-theta and an XRPD pattern as depicted in FIG. 6. The present disclosure also provides the use of the solid state form of Midostaurin for preparing other solid state forms of Midostaurin and solid state forms thereof.

Crystalline Form IX of Midostaurin may be a solvate. Crystalline Form IX may be a Methyl-acetate solvate.

PXRD peak list for crystalline Form IX of Midostaurin is shown in table 5.

TABLE 5
peak position (degrees two theta ±
0.2 degrees two-theta)

6.3
8.1
9.9
11.6
11.9
12.6
13.3
13.7
14.6
16.3
17.0
17.8
18.4
18.8
19.4
20.0
20.6
22.8
23.7
24.0
24.6
25.2
26.1
26.9
27.6
28.0
28.5
29.0
30.2
31.3
31.8
32.4
32.8
34.0
35.6

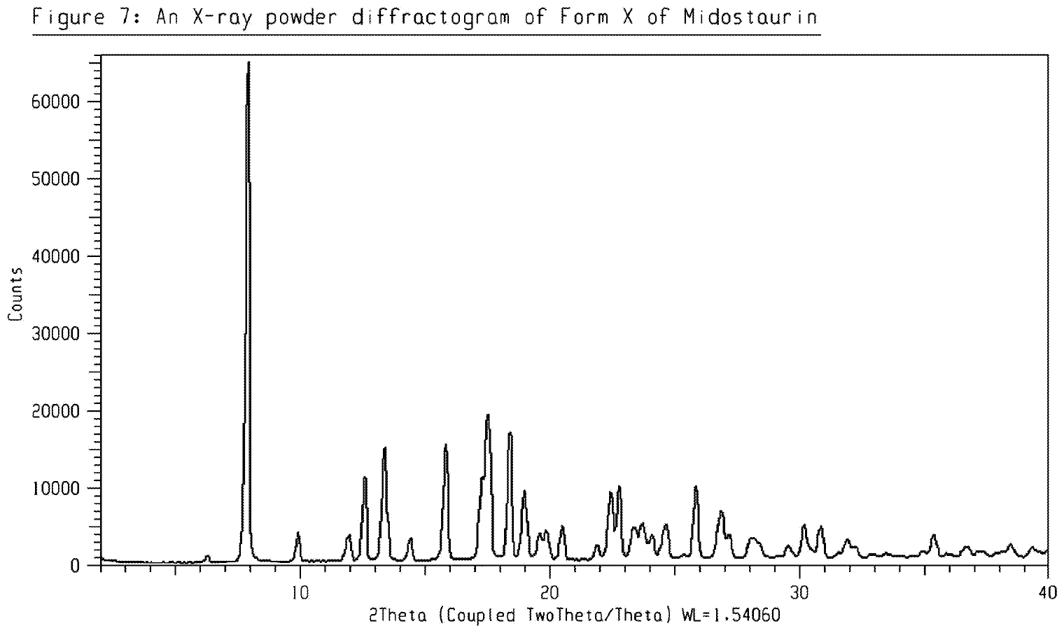

The present disclosure comprises a crystalline form of Midostaurin designated as Form X. The crystalline Form X of Midostaurin can be characterized by data selected from one or more of the following: an XRPD pattern having peaks at 7.9, 9.9, 12.6, 13.4 and 15.9 degrees 2-theta±0.2 degrees 2-theta; an XRPD pattern as depicted in FIG. 7; and combinations of these data.

Crystalline Form X of Midostaurin may be further characterized by the XRPD pattern having peaks at 7.9, 9.9, 12.6, 13.4 and 15.9 degrees 2-theta±0.2 degrees 2-theta, and also having one, two, three, four or five additional peaks selected from 17.6, 18.4, 19.0, 22.5 and 25.9 degrees 2-theta±0.2 degrees 2-theta.

Crystalline Form X of Midostaurin may be characterized by each of the above characteristics alone/or by all possible combinations, e.g. by XRPD pattern having peaks at 7.9, 9.9, 12.6, 13.4 and 15.9 degrees 2-theta±0.2 degrees 2-theta and an XRPD pattern as depicted in FIG. 7. The present disclosure also provides the use of the solid state form of Midostaurin for preparing other solid state forms of Midostaurin and solid state forms thereof.

Crystalline Form X of Midostaurin may be a solvate. Crystalline Form X may be an acetone solvate.

PXRD peak list for crystalline Form X of Midostaurin is shown in table 6.

TABLE 6
peak position (degrees two theta ±
0.2 degrees two-theta)

6.3
7.9
9.9
11.9
12.6
13.4
14.4
15.9
17.6
18.4
19.0
19.6
19.9
20.5
21.9
22.5
22.8
23.4
23.8
24.1
24.7
25.9
26.9
30.2
30.9

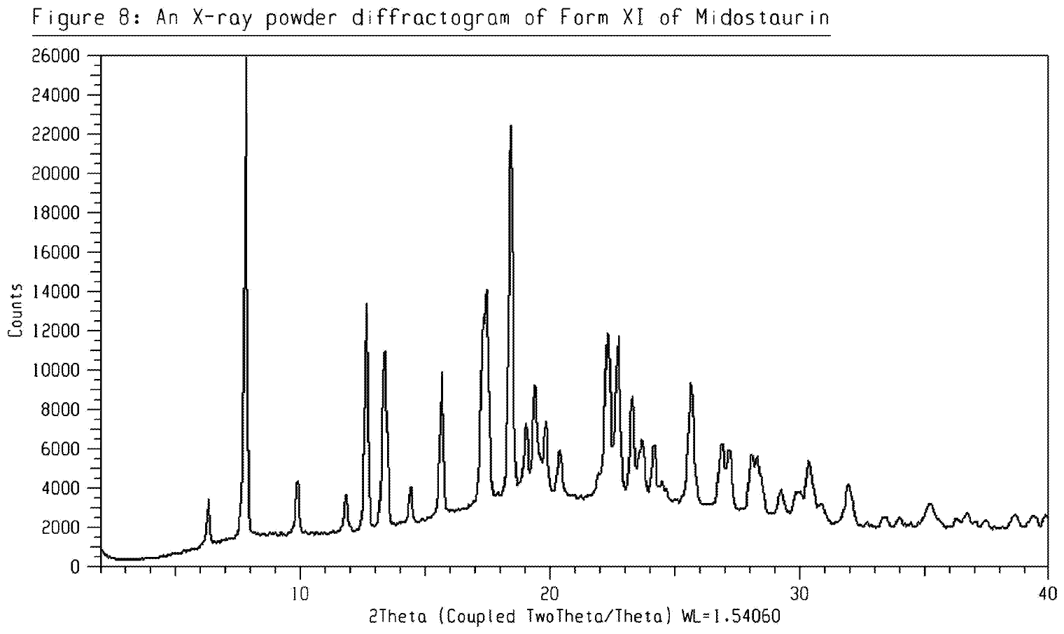

The present disclosure comprises a crystalline form of Midostaurin designated as Form XI. The crystalline Form XI of Midostaurin can be characterized by data selected from one or more of the following: an XRPD pattern having peaks at 7.8, 12.7, 13.4, 15.7 and 18.5 degrees 2-theta±0.2 degrees 2-theta; an XRPD pattern as depicted in FIG. 8; and combinations of these data.

Crystalline Form XI of Midostaurin may be further characterized by the XRPD pattern having peaks at 7.8, 12.7, 13.4, 15.7 and 18.5 degrees 2-theta±0.2 degrees 2-theta, and also having one, two, three, four or five additional peaks selected from 6.3, 9.9, 17.5, 22.4 and 22.8 degrees 2-theta±0.2 degrees 2-theta.

Crystalline Form XI of Midostaurin may be characterized by each of the above characteristics alone/or by all possible combinations, e.g. by XRPD pattern having peaks at 7.8, 12.7, 13.4, 15.7 and 18.5 degrees 2-theta±0.2 degrees 2-theta and an XRPD pattern as depicted in FIG. 8. The present disclosure also provides the use of the solid state form of Midostaurin for preparing other solid state forms of Midostaurin and solid state forms thereof.

Crystalline Form XI of Midostaurin may be a solvate. Crystalline Form XI may be an ethyl-formate solvate.

PXRD peak list for crystalline Form XI of Midostaurin is shown in table 7.

TABLE 7
peak position (degrees two theta ±
0.2 degrees two-theta)

6.3
7.8
9.9
11.8
12.7
13.4
14.4
15.7
17.5
18.5
19.1
19.4
19.9
20.4
22.4
22.8
23.3
23.7
24.2
25.7
26.9
27.2
28.2
28.3
29.3
30.4
32.0

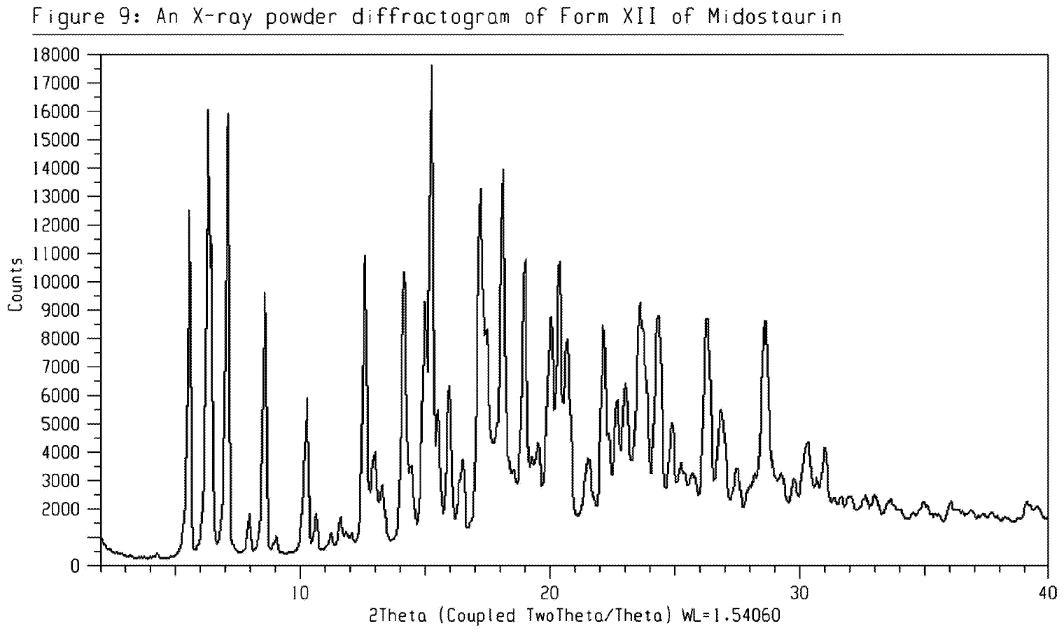

The present disclosure comprises a crystalline form of Midostaurin designated as Form XII. The crystalline Form XII of Midostaurin can be characterized by data selected from one or more of the following: an XRPD pattern having peaks at 5.6, 6.4, 7.1, 8.6 and 10.3 degrees 2-theta±0.2 degrees 2-theta; an XRPD pattern as depicted in FIG. 9; and combinations of these data.

Crystalline Form XII of Midostaurin may be further characterized by the XRPD pattern having peaks at 5.6, 6.4, 7.1, 8.6 and 10.3 degrees 2-theta±0.2 degrees 2-theta, and also having one, two, three, four or five additional peaks selected from 12.6, 14.2, 15.3, 18.2 and 19.0 degrees 2-theta±0.2 degrees 2-theta.

Crystalline Form XII of Midostaurin may be characterized by each of the above characteristics alone/or by all possible combinations, e.g. by XRPD pattern having peaks at 5.6, 6.4, 7.1, 8.6 and 10.3 degrees 2-theta±0.2 degrees 2-theta and an XRPD pattern as depicted in FIG. 9. The present disclosure also provides the use of the solid state form of Midostaurin for preparing other solid state forms of Midostaurin and solid state forms thereof.

Crystalline Form XII of Midostaurin may be a solvate. Crystalline Form XII may be an Isopropyl-acetate solvate.

PXRD peak list for crystalline Form XII of Midostaurin is shown in table 8.

TABLE 8
peak position (degrees two theta ±
0.2 degrees two-theta)

5.6
6.4
7.1
8.0
8.6
9.1
10.3
10.7
12.6
13.0
13.3
14.2
15.1
15.3
15.6
16.0
16.5
17.3
18.2
19.0
20.1
20.4
20.7
21.6
22.2
22.7
23.1
23.7
24.4
24.9
26.3
26.9
27.5
28.6
30.3
31.0

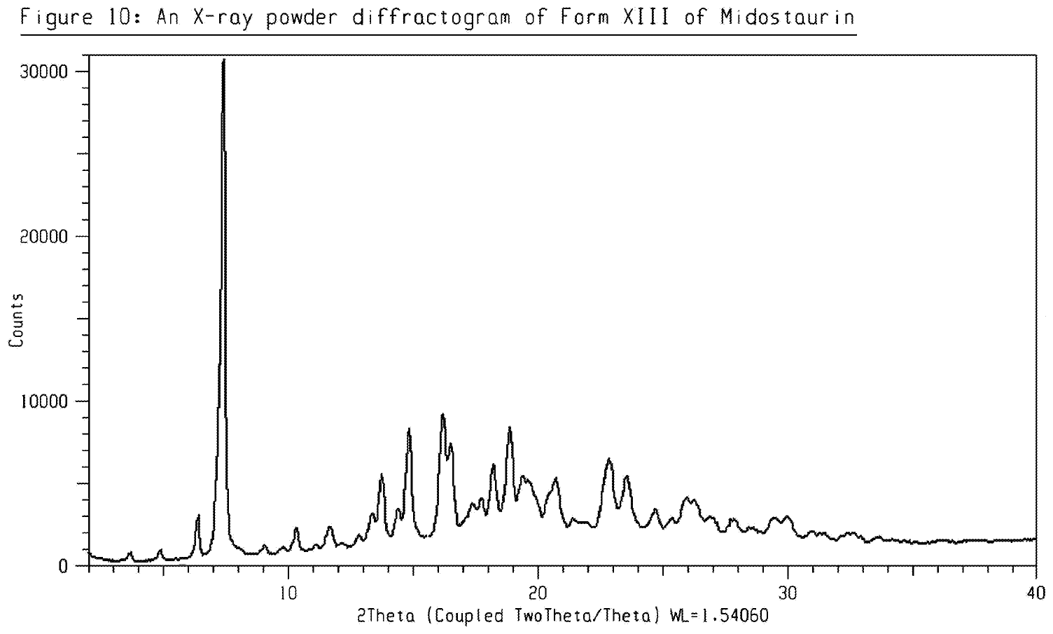

The present disclosure comprises a crystalline form of Midostaurin designated as Form XIII. The crystalline Form XIII of Midostaurin can be characterized by data selected from one or more of the following: an XRPD pattern having peaks at 6.4, 7.4, 13.7, 14.8 and 18.9 degrees 2-theta±0.2 degrees 2-theta; an XRPD pattern as depicted in FIG. 10; and combinations of these data.

Crystalline Form XIII of Midostaurin may be further characterized by the XRPD pattern having peaks at 6.4, 7.4, 13.7, 14.8 and 18.9 degrees 2-theta±0.2 degrees 2-theta, and also having one, two, three, four or five additional peaks selected from 10.3, 11.7, 18.2, 20.7 and 22.8 degrees 2-theta±0.2 degrees 2-theta.

Crystalline Form XIII of Midostaurin may be characterized by each of the above characteristics alone/or by all possible combinations, e.g. by XRPD pattern having peaks at 6.4, 7.4, 13.7, 14.8 and 18.9 degrees 2-theta±0.2 degrees 2-theta and an XRPD pattern as depicted in FIG. 10. The present disclosure also provides the use of the solid state form of Midostaurin for preparing other solid state forms of Midostaurin and solid state forms thereof.

Crystalline Form XIII of Midostaurin may be a solvate. Crystalline Form XIII may be a diethyl-carbonate solvate.

PXRD peak list for crystalline Form XIII of Midostaurin is shown in table 9.

TABLE 9
peak position (degrees two theta ±
0.2 degrees two-theta)

3.7
4.9
6.4
7.4
9.1
9.8
10.3
11.1
11.7
12.1
12.8
13.4
13.7
14.4
14.8
16.2
16.5
17.4
17.7
18.2
18.9
19.4
20.7
22.8
23.5
24.7
25.9
26.2
26.9
27.8
29.4
29.9

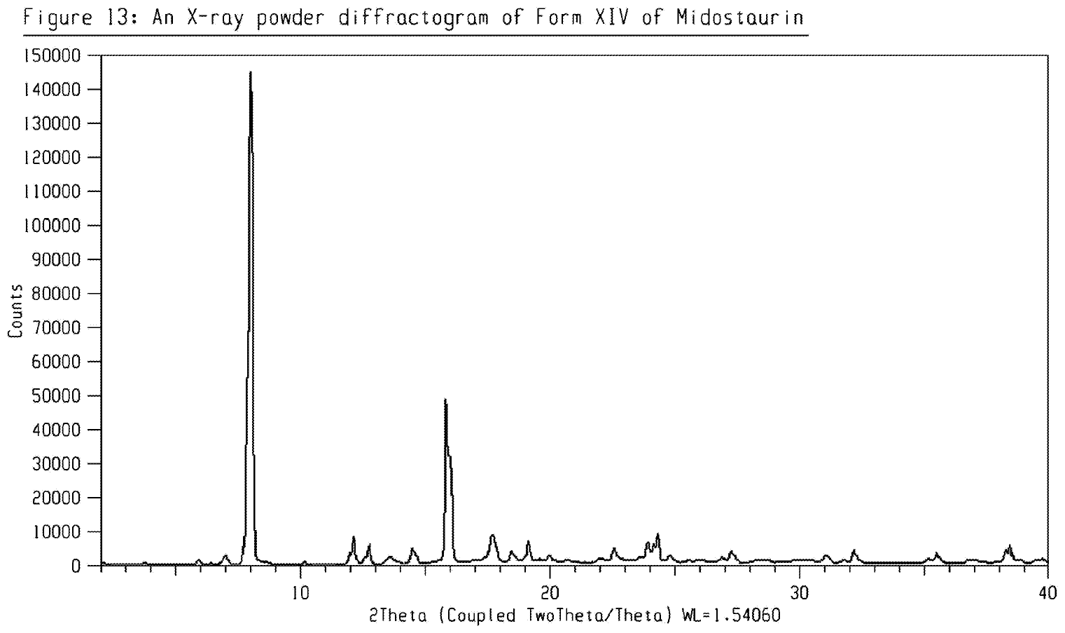

The present disclosure comprises a crystalline form of Midostaurin designated as Form XIV. The crystalline Form XIV of Midostaurin can be characterized by data selected from one or more of the following: an XRPD pattern having peaks 8.0, 13.5, 14.5, 15.9 and 17.7 degrees 2-theta±0.2 degrees 2-theta; an XRPD pattern as depicted in FIG. 13; and combinations of these data.

Crystalline Form XIV of Midostaurin may be further characterized by the XRPD pattern having peaks at 8.0, 13.5, 14.5, 15.9 and 17.7 degrees 2-theta±0.2 degrees 2-theta, and also having one, two, three, four or five additional peaks selected from 18.5, 19.1, 22.5, 24.8 and 27.3 degrees 2-theta±0.2 degrees 2-theta.

Crystalline Form XIV of Midostaurin may be characterized by each of the above characteristics alone/or by all possible combinations, e.g. by XRPD pattern having peaks at 8.0, 13.5, 14.5, 15.9 and 17.7 degrees 2-theta±0.2 degrees 2-theta and an XRPD pattern as depicted in FIG. 13. The present disclosure also provides the use of the solid state form of Midostaurin for preparing other solid state forms of Midostaurin and solid state forms thereof.

Crystalline Form XIV of Midostaurin may be a solvate. Crystalline Form XIV may be a benzonitrile solvate.

PXRD peak list for crystalline Form XIV of Midostaurin is shown in table 10.

TABLE 10
peak position (degrees two theta ±
0.2 degrees two-theta)

3.7
5.9
7.0
8.0
12.1
12.7
13.5
14.5
15.9
17.7
18.5
19.1
22.5
23.9
24.3
24.8
27.3
31.0
32.1

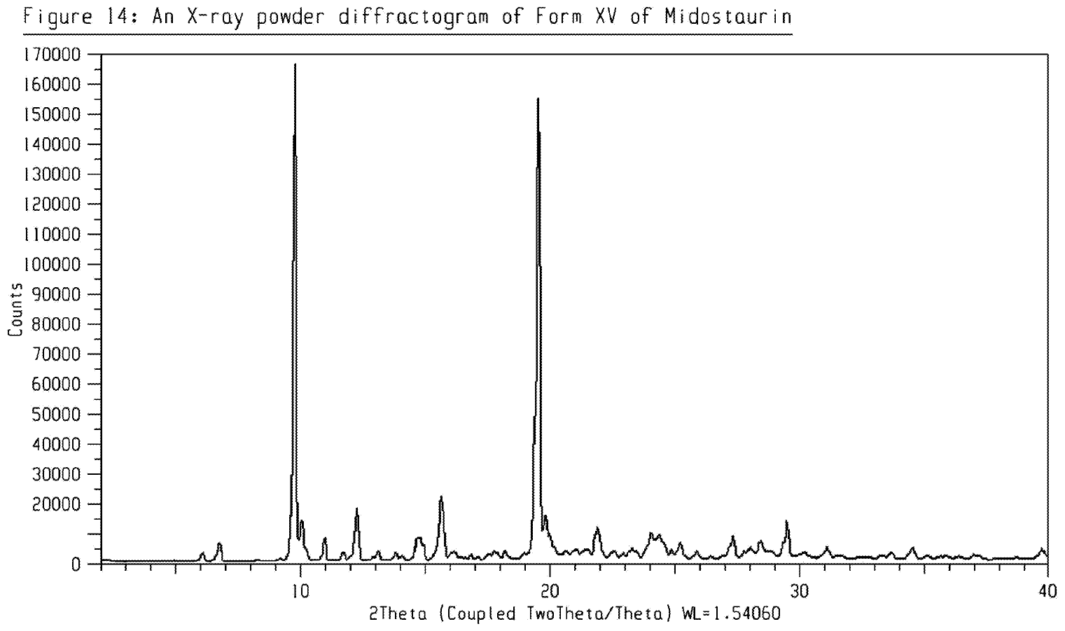

The present disclosure comprises a crystalline form of Midostaurin designated as Form XV. The crystalline Form XV of Midostaurin can be characterized by data selected from one or more of the following: an XRPD pattern having peaks 9.8, 12.3, 15.7, 19.6 and 21.9 degrees 2-theta±0.2 degrees 2-theta; an XRPD pattern as depicted in FIG. 14; and combinations of these data.

Crystalline Form XV of Midostaurin may be further characterized by the XRPD pattern having peaks at 9.8, 12.3, 15.7, 19.6 and 21.9 degrees 2-theta±0.2 degrees 2-theta, and also having one, two, three, four or five additional peaks selected from 6.7, 11.0, 24.1, 27.4, and 29.5 degrees 2-theta±0.2 degrees 2-theta.

Crystalline Form XV of Midostaurin may be characterized by each of the above characteristics alone/or by all possible combinations, e.g. by XRPD pattern having peaks at 9.8, 12.3, 15.7, 19.6 and 21.9 degrees 2-theta±0.2 degrees 2-theta and an XRPD pattern as depicted in FIG. 14. The present disclosure also provides the use of the solid state form of Midostaurin for preparing other solid state forms of Midostaurin and solid state forms thereof.

Crystalline Form XV of Midostaurin may be a solvate. Crystalline Form XV may be a n-butyl acetate solvate.

PXRD peak list for crystalline Form XV of Midostaurin is shown in table 11.

TABLE 11
peak position (degrees two theta ±
0.2 degrees two-theta)

6.1
6.7
9.8
10.0
11.0
11.7
12.3
14.7
15.7
19.6
19.8
21.9
24.1
24.4
25.3
27.4
28.5
29.5

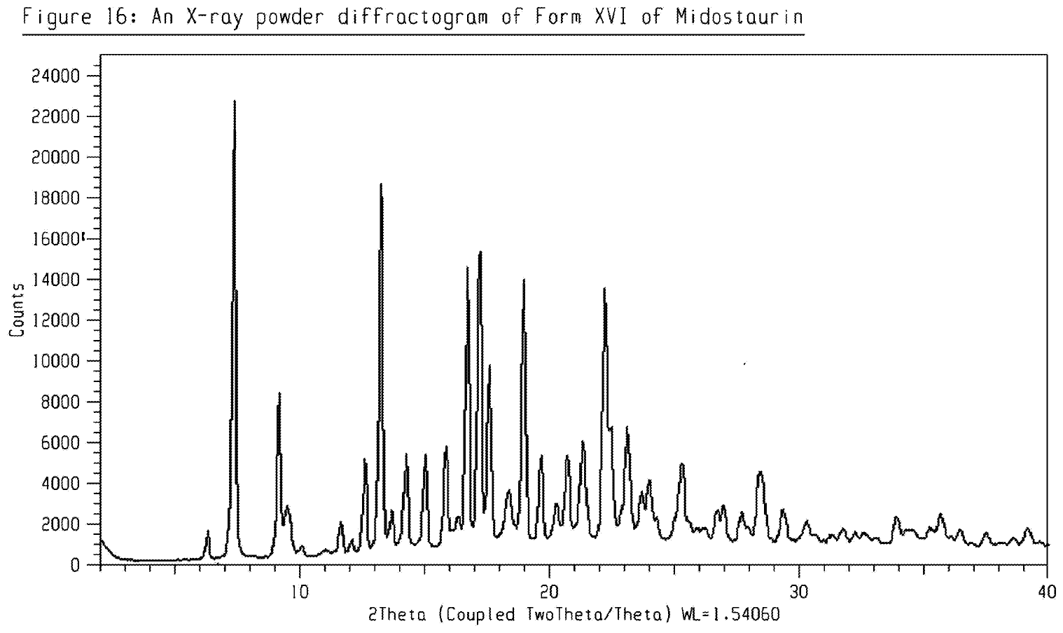

The present disclosure comprises a crystalline form of Midostaurin designated as Form XVI. The crystalline Form XVI of Midostaurin can be characterized by data selected from one or more of the following: an XRPD pattern having peaks 7.3, 9.1, 13.2, 16.7 and 17.2 degrees 2-theta±0.2 degrees 2-theta; an XRPD pattern as depicted in FIG. 16; and combinations of these data.

Crystalline Form XVI of Midostaurin may be further characterized by the XRPD pattern having peaks at 7.3, 9.1, 13.2, 16.7 and 17.2 degrees 2-theta±0.2 degrees 2-theta, and also having one, two, three, four or five additional peaks selected from 12.6, 14.2, 15.0, 17.6 and 19.0 degrees 2-theta±0.2 degrees 2-theta.

Crystalline Form XVI of Midostaurin may be characterized by each of the above characteristics alone/or by all possible combinations, e.g. by XRPD pattern having peaks at 7.3, 9.1, 13.2, 16.7 and 17.2 degrees 2-theta±0.2 degrees 2-theta and an XRPD pattern as depicted in FIG. 16. The present disclosure also provides the use of the solid state form of Midostaurin for preparing other solid state forms of Midostaurin and solid state forms thereof.

Crystalline Form XVI of Midostaurin may be a solvate. Crystalline Form XVI may be a tert-butanol solvate.

PXRD peak list for crystalline Form XVI of Midostaurin is shown in table 12.

Peak position (degrees two theta ±
0.2 degrees two-theta)

6.3
7.3
9.1
9.5
11.6
12.1
12.6
13.2
13.7
14.2
15.0
15.8
16.7
17.2
17.6
18.4
19.0
19.7
20.3
20.7
21.3
22.2
22.4
23.1
23.7
24.0
25.3
27.7
28.5
29.4

The present disclosure also provides uses of the solid state forms of Midostaurin for preparing other solid state forms of Midostaurin.

The present disclosure further encompasses processes for preparing other solid state forms of Midostaurin. The process comprises preparing any one of the solid state forms of Midostaurin of the present disclosure, and converting it to other solid state forms of Midostaurin.

In another embodiment, the present disclosure encompasses the above described solid state form of Midostaurin for use in the preparation of pharmaceutical compositions and/or formulations, optionally for the treatment of Acute Myelogenous Leukemia (AML) and Aggressive Systemic Mastocytosis.

In another embodiment, the present disclosure encompasses the use of the above described solid state forms of Midostaurin for the preparation of pharmaceutical compositions and/or formulations. The present disclosure also provides the solid state forms of Midostaurin of the present disclosure for use in the preparation of pharmaceutical compositions and/or formulations.

The present disclosure further provides pharmaceutical compositions comprising any one or a mixture of the solid state forms of Midostaurin according to the present disclosure.

In yet another embodiment, the present disclosure encompasses pharmaceutical formulations comprising any one or a mixture of the solid state forms of Midostaurin; and at least one pharmaceutically acceptable excipient.

The present disclosure encompasses processes to prepare said formulations of Midostaurin comprising combining any one or a mixture of the solid state forms of Midostaurin and at least one pharmaceutically acceptable excipient.

The solid state forms of Midostaurin as defined herein, as well as the pharmaceutical compositions or formulations thereof, at least can be used as medicaments, particularly for the treatment of Acute Myelogenous Leukemia (AML) and Aggressive Systemic Mastocytosis.

The present disclosure also provides methods of treating Acute Myelogenous Leukemia (AML) and Aggressive Systemic Mastocytosis; comprising administering a therapeutically effective amount of any one or a mixture of the solid state forms of Midostaurin of the present disclosure, or at least one of the above pharmaceutical compositions or formulations, to a subject suffering from Acute Myelogenous Leukemia (AML) and Aggressive Systemic Mastocytosis, or otherwise in need of the treatment.

The present disclosure also provides use of the solid state forms of Midostaurin, or at least one of the above pharmaceutical compositions or formulations for the manufacture of a medicament for treating Acute Myelogenous Leukemia (AML) and Aggressive Systemic Mastocytosis.

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### Analytical Methods

X-Ray Powder Diffraction Method:

BRUKER D8 Advance X-ray powder diffractometer, CuKα radiation (λ=1.5418 Å); Lynxeye XE detector, low amount PMMA sample holder with zero background plate was used. Prior to analysis, the dry samples were gently ground by means of mortar and pestle in order to obtain a fine powder. The ground sample was adjusted into a cavity of the sample holder and the surface of the sample was smoothed by means of a cover glass.

a. Measurement parameters:
b. Sample: Spin mode, rotation speed 30 rpm;
c. Scan range: 2-40 degrees 2-theta;
d. Scan mode: continuous;
e. Step size: 0.05±0.005 degrees;
f Time per step: 0.5 sec;
g. Divergence slit: V20

The accuracy of peak positions is defined as ±0.2 degrees two theta due to experimental differences like instrumentations, sample preparations etc.

### FT-IR Spectroscopy

Perkin-Elmer Spectrum One FT-IR Spectrometer, at 4 cm⁻¹ resolution with 16 scans, in the range of 4000-400 cm⁻¹. Samples were analysed in KBr disk. The spectra were recorded using an empty cell as a background.

### EXAMPLES

The starting material of the synthesis, (9S,10R,11R,13R)-2,3,10,11,12,13-Hexahydro-10-methoxy-9-methyl-11-(methylamino)-9,13-epoxy-1H,9H-diindolo[1,2,3-gh:3′,2′,1′-lm]pyrrolo[3,4-j][1,7]benzodiazonin-1-one, also known as staurosporine, can be produced by any process described in the literature, for example in Exp 1 and 2 in U.S. Pat. No. 4,107,297.

### Example 1: Synthesis of Crude Midostaurin

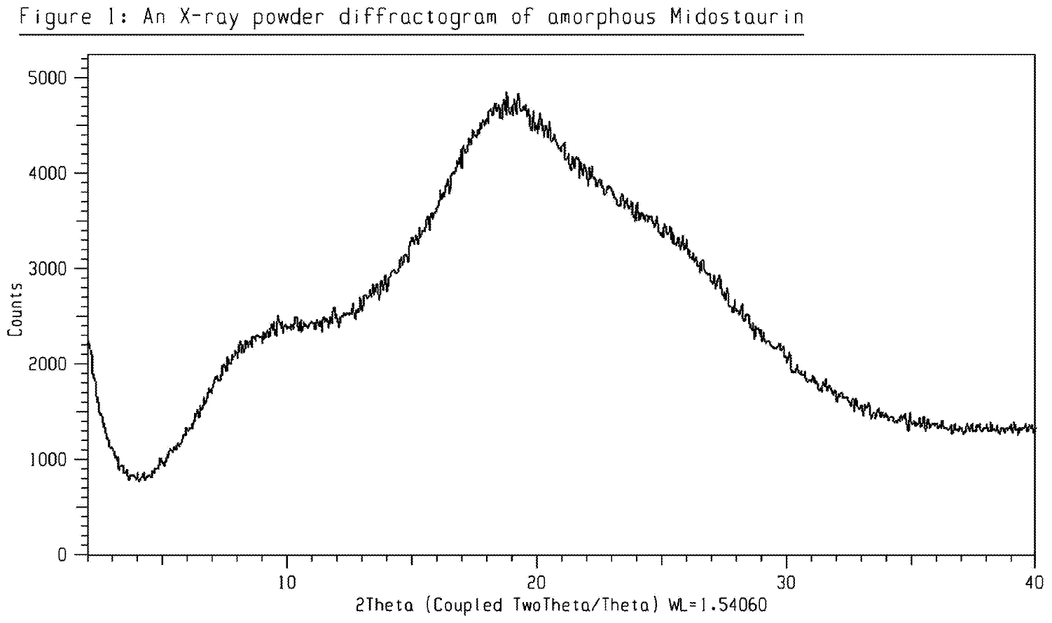

5 g from the obtained product staurosporine was dissolved in dimethyl formamide (DMF). Base was added, which can be N,N-diisopropyl-ethyl-amine (2.70 mL) or Triethyl-amine (2.20 mL) and benzoyl-chloride (1.45 mL) was dropped into the mixture. After reaction completion the mixture was diluted with iBuOAc (625 mL), washed with saturated NaHCO₃ solution and water. Crude Midostaurin was precipitated after evaporation of organic phase by addition of n-Heptane (1500 mL) at room temperature. The suspension was cooled to 5° C. for 18 hours, filtered and dried at 55° C., under reduced pressure to give N-[(9S,10R,11R,13R)-2,3,10,11,12,13-Hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide amorphous solid. The obtained product was characterized by XRD-PXRD pattern of amorphous Midostaurin is shown in FIG. 1.

### Example 1a: Synthesis of Crude Midostaurin

210 g from the obtained product staurosporine was dissolved in dimethyl formamide (DMF, 5.25 L). 61.5 ml Triethyl-amine was added and benzoyl-chloride (41 mL) was dropped into the mixture. After reaction completion the mixture was diluted with iso-Buthyl-acetate (iBuOAc, 26.25 L), washed with saturated NaHCO₃ solution and water. Crude Midostaurin was precipitated after evaporation of organic phase to 1/10 volume and addition of n-Heptane (45 L) at room temperature (25° C.). The suspension was cooled to 5° C. for 18 hours, filtered and dried at 60° C., under reduced pressure to give N-[(9S,10R,11R,13R)-2,3,10,11,12,13-Hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide.

### Example 2a: Purification of Crude Midostaurin

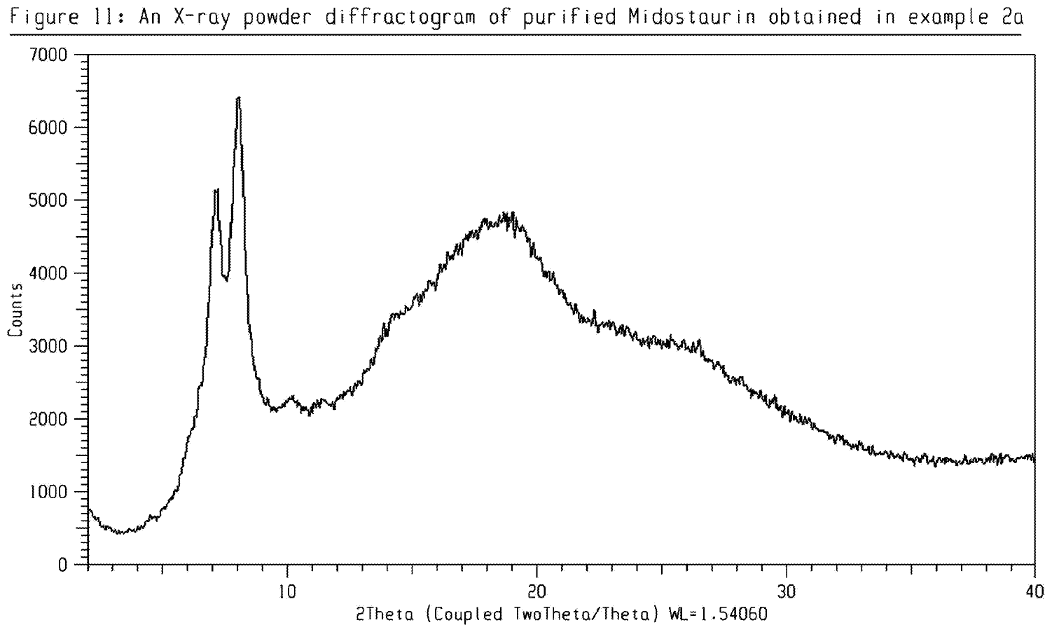

3 g crude amorphous Midostaurin (from example 1) was dissolved in 150 mL Toluene:Methanol (9:1) and loaded to a column packed with YMC-S50 normal phase Silica gel. Eluent was Toluene: Methanol mixture (95:5). Fractions having HPLC purity more than 99.5 Area % was combined and evaporated to dryness. The resulted solid was suspended in 70 mL n-Heptane, filtered and dried in vacuum at 80° C., 20 mbar for 13 hours to give purified Midostaurin having HPLC purity of about 99.5 Area %. The obtained product was characterized by XRD-PXRD pattern is shown in FIG. 11.

### Example 2b: Purification of Crude Midostaurin

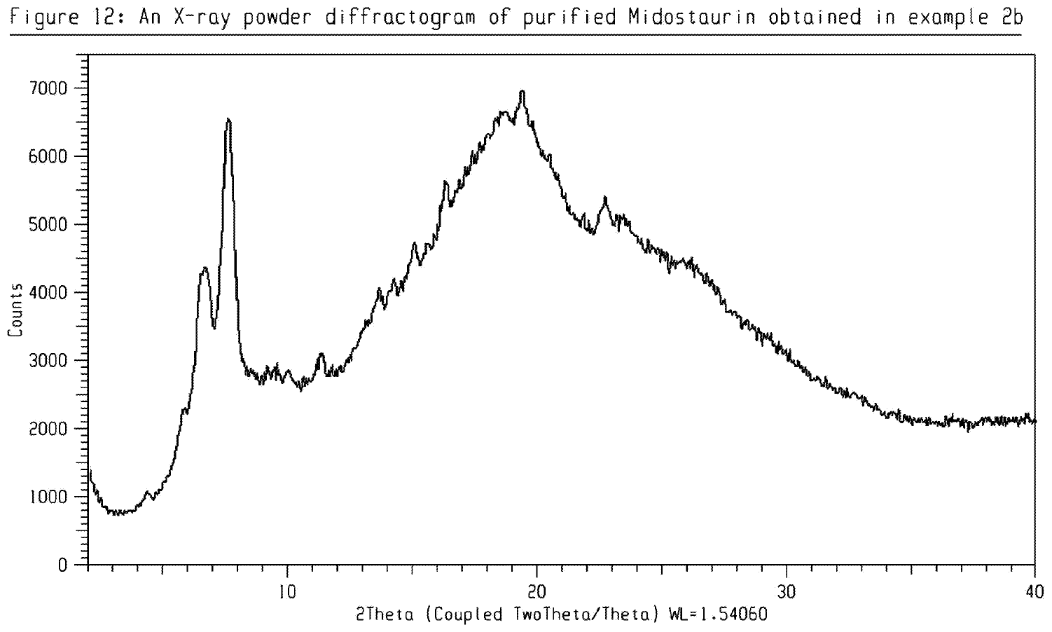

3 g crude amorphous Midostaurin (from example 1) was dissolved in 150 ml Toluene:Methanol (9:1) and loaded to a column packed with YMC-S50 normal phase Silica gel. Eluent was Toluene: Methanol mixture (95:5). Fractions having HPLC purity more than 99.5 Area % was combined, evaporated to dryness and further dried in vacuum at 60° C., 20 mbar for 4 days to give purified Midostaurin having HPLC purity of about 99.51 Area %. The obtained product was characterized by XRD-PXRD pattern is shown in FIG. 12.

### Example 2c: Purification of Crude Midostaurin

128 g crude Midostaurin (from example 1a) was dissolved in 5 L Toluene: Methanol (9:1) and loaded to a column packed with YMC-S50 normal phase Silica gel. Eluent was Toluene: Methanol mixture (95:5). Fractions having HPLC purity more than 99.5 Area % was combined and evaporated to dryness.

32 g from the evaporated solid was dissolved in 280 ml Dimethyl-formamide at 25° C., and filtered on 0.22 ™ filter paper. The filtrate was added to 1700 ml pre-cooled water (2° C.), stirred for 5 minutes, and filtered on glass filter having pore size: 4. The filtered solid washed two times with 850 ml water by stirring for 30 minutes, and filtered. The solid amorphous Midostaurin was dried at 80° C. in vacuum (100 mbar), for 2×24 hours and found to be amorphous Midostaurin according to FIG. 1. HPLC purity was: 99.61 Area %.

### Example 3: Preparation of Midostaurin Form V

1 g purified Midostaurin (from example 2a) (HPLC purity: 99.53 Area %) was dissolved in 300 mL Ethyl-acetate at 40° C. and filtered on a glass filter. The solution was evaporated to about 1/10 volume under reduced pressure at 50° C. The concentrate was dropped into 600 mL n-Heptane within 5 minutes. The suspension was cooled to 5° C. for 18 hours, filtered and dried at 50° C., under reduced pressure (20 mbar) for 8 hours to give Midostaurin Form V. The obtained product was characterized by XRD-PXRD pattern is shown in FIG. 2.

### Example 4: Preparation of Midostaurin Form V

1 g purified Midostaurin (from example 2a) (HPLC purity: 99.53 Area %) was disolved in 300 mL Ethyl-acetate at 40° C. and filtered on a glass filter. The solution was evaporated to about 1/10 volume under reduced pressure at 50° C. 600 mL n-Heptane was dropped into the concentrate within 60 minutes. The suspension was cooled to 5° C. for 18 hours, filtered and dried at 50° C., under reduced pressure (20 mbar) for 8 hours to give Midostaurin Form V. PXRD pattern of Form V of Midostaurin is shown in FIG. 2.

### Example 5: Preparation of Midostaurin Form VI

0.2 g of starting material (from example 2b) (HPLC purity: 99.51 Area %) was slurried in isopropyl alcohol (28 ml) at 20-25° C., for 24 hours with magnetic stirrer. After stirring the suspension was filtered. The product was dried for 26 h at 60° C. in vacuum (˜100 mbar). The sample was analyzed by XRPD and the XRPD pattern of Form VI of Midostaurin is shown in FIG. 3.

### Example 5a: Preparation of Midostaurin Form VI

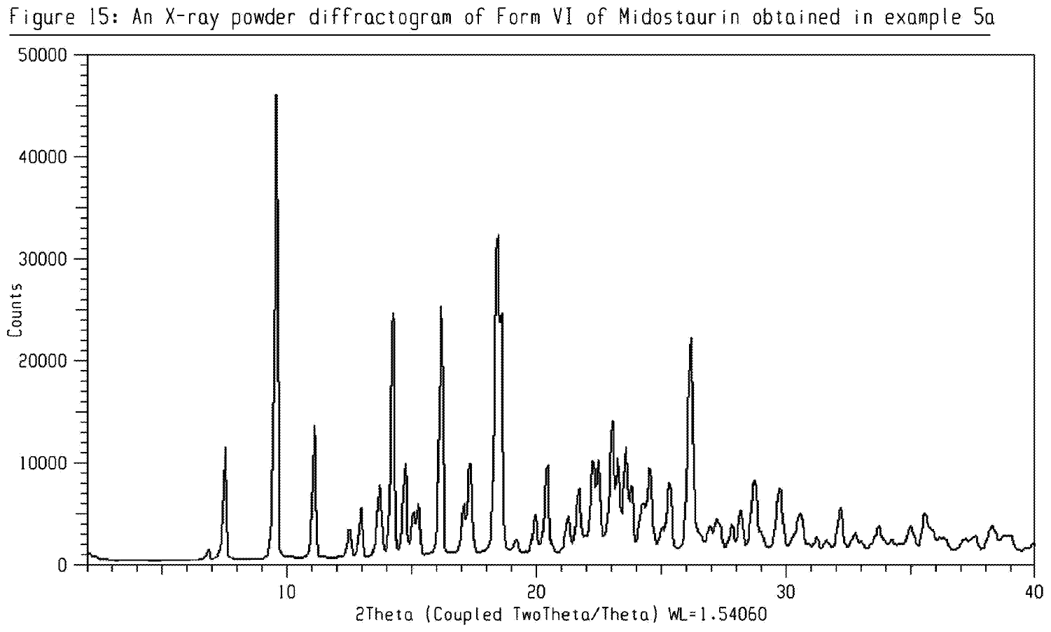

0.2 g of starting material (from example 2b) (HPLC purity: 99.51 Area %) was dissolved in methanol (20 ml) at 20-25° C., solvent was allowed to evaporate at 20-25° C. for 7 days. The obtained product was characterized by X-ray powder diffraction to obtain crystalline Form VI having the diffractogram as shown in FIG. 15.

### Example 5b: Preparation of Midostaurin Form VI

0.2 g of starting material (from example 2b) (HPLC purity: 99.51 Area %) was dissolved in ethanol (20 ml) at 20-25° C., solvent was allowed to evaporate at 20-25° C. for 8 days. The obtained product was characterized by X-ray powder diffraction to obtain the crystalline Form VI.

### Example 5c: Preparation of Midostaurin Form VI

0.2 g of starting material (from example 2b) (HPLC purity: 99.51 Area %) was dissolved was dissolved in 96% ethanol (26 ml) at 20-25° C., solvent was allowed to evaporate at 20-25° C. for 16 days. The obtained product was characterized by X-ray powder diffraction to obtain the crystalline Form VI.

### Example 5d: Preparation of Midostaurin Form VI

0.2 g of starting material (from example 2b) (HPLC purity: 99.51 Area %) was dissolved was dissolved in n-propanol (20 ml) at 20-25° C., stirred with magnetic stirrer for 6 days, solvent was allowed to evaporate to about ½ volume at 20-25° C., then the suspension was filtered. The product was dried for 23 h at 20-25° C. in atmospheric pressure. The obtained product was characterized by X-ray powder diffraction to obtain the crystalline Form VI.

### Example 5e: Preparation of Midostaurin Form VI

0.2 g of starting material (from example 2b) (HPLC purity: 99.51 Area %) was slurried in 2-butanol (20 ml) at 20-25° C., for 4 days with magnetic stirrer. After stirring the suspension was filtered. The product was dried for 23 h at 60° C. in vacuum (100 mbar). The obtained product was characterized by X-ray powder diffraction to obtain the crystalline Form VI.

### Example 5f: Preparation of Midostaurin Form VI

0.2 g of starting material (from example 2b) (HPLC purity: 99.51 Area %) was slurried in toluene (1 ml) at 20-25° C., then methanol (4 ml) was added and stirred with magnetic stirrer for 1 day. After stirring the suspension was filtered. The product was dried for 2 h at 20-25° C. in atmospheric pressure. The obtained product was characterized by X-ray powder diffraction to obtain the crystalline Form VI.

### Example 5g: Preparation of Midostaurin Form VI

10 g Midostaurin (from example 2c) was slurried in 200 ml methanol at 60° C. for 10 minutes. The suspension was cooled to 20-25° C. for 1 hour, stirred for 22 hours at 20-25° C., filtered on G4 glass filter and dried at 80° C. for 27.5 hours under reduced pressure (80 mbar). The obtained product was characterized by X-ray powder diffraction to obtain the crystalline Form VI.

### Example 5h: Preparation of Midostaurin Form VI

4 g of Midostaurin (from example 2c) was dissolved in 20 ml dimethyl-formamide at 20-25° C. 400 ml methanol-water 80:20 mixture was dropped into the solution within 30 minutes. The suspension was stirred at 20-25° C. for 24 hours, filtered on G4 glass filter, washed with 4×160 ml methanol-water 80:20 mixture and dried at 80° C. for 44 hours under reduced pressure (100 mbar). The obtained product was characterized by X-ray powder diffraction to obtain the crystalline Form VI.

### Example 6: Preparation of Midostaurin Form VII

0.2 g of starting material (from example 2b) (HPLC purity: 99.51 Area %) was dissolved in methyl isobutyl ketone (MIBUK) (10 ml) at 20-25° C., solvent was allowed to evaporate at 20-25° C. for 4 days. The sample was analyzed by XRPD and the XRPD pattern of Form VII of Midostaurin is shown in FIG. 4.

### Example 7: Preparation of Midostaurin Form VIII

0.2 g of starting material (from example 2b) (HPLC purity: 99.51 Area %) was slurried in 4-heptanone (20 ml) at 20-25° C., for 3 days with a magnetic stirrer. After stirring, the suspension was filtered. The product was dried for 23 h at 20-25° C. in atmospheric pressure. The sample was analyzed by XRPD and the XRPD pattern of Form VIII of Midostaurin is shown in FIG. 5.

### Example 8: Preparation of Midostaurin Form IX

0.2 g of starting material (from example 2b) (HPLC purity: 99.51 Area %) was slurried in methyl acetate (20 ml) at 20-25° C., for 1 day with magnetic stirrer. Then, solvent was allowed to evaporate at 20-25° C. for 3 days. The sample was analyzed by XRPD and the XRPD pattern of Form IX of Midostaurin is shown in FIG. 6.

### Example 8a: Preparation of Midostaurin Form IX

0.2 g of starting material (from example 2b) (HPLC purity: 99.51 Area %) was dissolved in dimethyl carbonate (26 ml) at 20-25° C., stirred with magnetic stirrer for 1 day, solvent was allowed to evaporate to about ½ volume at 20-25° C., then the suspension was filtered. The product was dried for 23 h at 20-25° C. in atmospheric pressure. The obtained product was characterized by X-ray powder diffraction to obtain the crystalline form IX having the same diffractogram as shown in FIG. 6.

### Example 9: Preparation of Midostaurin Form X

0.2 g of starting material (from example 2b) (HPLC purity: 99.51 Area %) was slurried in acetone (20 ml) at 20-25° C., for 20 h with magnetic stirrer. After stirring, the suspension was filtered. The product was dried for 21 h at 20-25° C. in atmospheric pressure. The sample was analyzed by XRPD and the XRPD pattern of Form X of Midostaurin is shown in FIG. 7.

### Example 10: Preparation of Midostaurin Form XI

0.2 g of starting material (from example 2b) (HPLC purity: 99.51 Area %) was slurried in ethyl formate (20 ml) at 20-25° C., for 1 day with magnetic stirrer. Then, solvent was allowed to evaporate at 20-25° C. for 5 days. The sample was analyzed by XRPD and the XRPD pattern of Form XI of Midostaurin is shown in FIG. 8.

### Example 10a: Preparation of Midostaurin Form XI

0.2 g of starting material (from example 2b) (HPLC purity: 99.51 Area %) was slurried in nitromethane (20 ml) at 20-25° C., for 4 days with magnetic stirrer, then the suspension was filtered. The product was dried for 23 h at 20-25° C. in atmospheric pressure. The obtained product was characterized by X-ray powder diffraction to obtain the crystalline Form XI having the same diffractogram as shown in FIG. 8.

### Example 11: Preparation of Midostaurin Form XII

0.2 g of starting material (from example 2b) (HPLC purity: 99.51 Area %) was slurried in isopropyl acetate (20 ml) at 20-25° C., for 1 day with magnetic stirrer. Then, solvent was allowed to evaporate at 20-25° C. for 4 days. The sample was analyzed by XRPD and the XRPD pattern of Form XII of Midostaurin is shown in FIG. 9.

### Example 12: Preparation of Midostaurin Form XIII

0.2 g of starting material (from example 2b) (HPLC purity: 99.51 Area %) was slurried in diethyl carbonate (20 ml) at 20-25° C., for 4 days with magnetic stirrer. After stirring, the suspension was filtered. The product was dried for 24 h at 20-25° C. in atmospheric pressure. The sample was analyzed by XRPD and the XRPD pattern of Form XIII of Midostaurin is shown in FIG. 10.

### Example 13: Preparation of Midostaurin Form XIV

0.2 g of starting material (from example 2b) (HPLC purity: 99.51 Area %) was dissolved in benzonitrile (3 ml) at 20-25° C. Acetone (25 ml) was added and then it was allowed to crystallize at −18° C. for 11 days. The formed solid material was filtered on G4 glass filter. The wet sample was analyzed by XRPD and the XRPD pattern of Form XIV of Midostaurin is shown in FIG. 13.

### Example 14: Preparation of Midostaurin Form XIV

0.2 g of starting material (from example 2b) (HPLC purity: 99.51 Area %) was dissolved in guaiacol (2 ml) at 20-25° C. Acetone (26 ml) was added and then it was allowed to crystallize at −18° C. for 12 days. The formed solid material was filtered on G4 glass filter. The wet sample was analyzed by XRPD to obtain the crystalline form XIV having the same diffractogram as shown in FIG. 13.

### Example 15: Preparation of Midostaurin Form XV

0.2 g of starting material (from example 2b) (HPLC purity: 99.51 Area %) was dissolved in n-butyl acetate (26 ml) at 20-25° C. Then, solvent was allowed to evaporate at 20-25° C. for 13 days. The sample was analyzed by XRPD and the XRPD pattern of Form XV of Midostaurin is shown in FIG. 14.

### Example 16: Preparation of Midostaurin Form XVI

0.5 g Midostaurin (obtained from Example 2c) was dissolved in 5 ml acetic acid at 20-25° C. 50 ml Ethyl-acetate was dropped into the solution. The solution was stirred at 20-25° C. for 23 hours. The solution was evaporated to about ½ volume under reduced pressure at 50° C. After evaporation 80 ml t-butanol was dropped into the solution at 20-25° C. and stirred at 20-25° C. for 19 hours. The suspension was filtered on G4 glass filter and dried at 80° C., under reduced pressure (150 mbar) for 2 hours to give Midostaurin Form XVI.

### Example 17: Preparation of Midostaurin Form XVI

0.5 g Midostaurin (obtained from Example 2c) was dissolved in 10 ml dichloromethane at 20-25° C. 90 ml tert-Butanol was dropped into the solution. The solution was stirred at 20-25° C. for 22 hours. The suspension was filtered on G4 glass filter and dried at 80° C., under reduced pressure (200 mbar) for 3 hours to give Midostaurin Form XVI.

1. Crystalline Form VI of Midostaurin, which is characterized by data selected from one of the following:
(i) an XRPD pattern having peaks at 7.5, 9.5, 11.0, 14.2 and 16.1 degrees 2-theta±0.2 degrees 2-theta;
(ii) an XRPD pattern as depicted in FIG. 3; and
combinations of any of (i) or (ii).

2. Crystalline Form VI of Midostaurin according to claim 1 which is characterized by data selected from one or more of the following:
(i) an XRPD pattern having peaks at 7.5, 9.5, 11.0, 14.2 and 16.1 degrees 2-theta±0.2 degrees 2-theta, and also having one, two, three, four or five additional peaks selected from 17.3, 18.4, 20.4, 23.0, and 26.1 degrees 2-theta±0.2 degrees 2-theta;
(ii) an FT-IR spectrum having one, two, three, four or more peaks selected from 3428, 1701, 1635, 1454, 1345, 1223, 1115, 1064, 823, 750 and 713 cm⁻¹±1 cm⁻¹;
(iii) an FT-IR spectrum as depicted in FIG. 17; and
combinations of any of (i)-(iii).

3. Crystalline Form VI of Midostaurin according to claim 1 wherein crystalline Form VI is a hydrate.

4. Crystalline Form VI of Midostaurin according to claim 1 characterized by a water content of between 1 to 5%.

5. Crystalline Form VI of Midostaurin according to claim 1 characterized by a water content of about 2%.

6. A pharmaceutical composition comprising a crystalline form according to claim 1.

7. Use of the crystalline form according to claim 1 in the preparation of pharmaceutical compositions and/or formulations.

8. A pharmaceutical formulation comprising a crystalline form according to claim 1, and at least one pharmaceutically acceptable excipient.

9. The crystalline form according to claim 1, for use as a medicament.

10. The crystalline form according to claim 1, for use in the treatment of Acute Myelogenous Leukemia (AML) or Aggressive Systemic Mastocytosis.

11. A method for treating Acute Myelogenous Leukemia (AML) or Aggressive Systemic Mastocytosis comprising administering a therapeutically effective amount of a crystalline form according to claim 1, to a subject suffering from said disorder, or otherwise in need of the treatment.

12. Use of the crystalline form according to claim 1, for the manufacture of a medicament for the treatment of Acute Myelogenous Leukemia (AML) or Aggressive Systemic Mastocytosis.

13. A process for preparing other solid state forms of Midostaurin comprising preparing a solid state form of Midostaurin according to claim 1 and converting it to another crystalline form of Midostaurin.

14. A pharmaceutical formulation comprising the pharmaceutical composition of claim 6, and at least one pharmaceutically acceptable excipient.

15. The pharmaceutical composition according to claim 6, for use as a medicament.

16. The pharmaceutical formulation according to claim 14, for use as a medicament.

17. The pharmaceutical composition according to claim 6, for use in the treatment of Acute Myelogenous Leukemia (AML) or Aggressive Systemic Mastocytosis.

18. The pharmaceutical formulation according to claim 14, for use in the treatment of Acute Myelogenous Leukemia (AML) or Aggressive Systemic Mastocytosis.

19. A method for treating Acute Myelogenous Leukemia (AML) or Aggressive Systemic Mastocytosis comprising administering a therapeutically effective amount of the pharmaceutical composition according to claim 6, to a subject suffering from said disorder, or otherwise in need of the treatment.

20. A method for treating Acute Myelogenous Leukemia (AML) or Aggressive Systemic Mastocytosis comprising administering a therapeutically effective amount of the of the pharmaceutical formulation according to claim 14, to a subject suffering from said disorder, or otherwise in need of the treatment.
